(19) United States
(12) Patent Application Publication
(10) Pub. No.: US 2026/0028301 A1
(43) Pub. Date: Jan. 29, 2026

(54) GROUP (VIII) CATALYSTS FOR GENERATION OF GREEN HYDROGEN AND ACETIC ACID FROM ETHANOL AND ITS MECHANISM THEREOF

(71) Applicant: INDIAN INSTITUTE OF TECHNOLOGY, GUWAHATI, Guwahati (IN)

(72) Inventors: Akshai KUMAR, Guwahati (IN); Eileen YASMIN, Kolkata (IN); Sunil DHOLE, Pune (IN); Tushar WAGH, Pune (IN); Vinay ARORA, Gurugram (IN)

(21) Appl. No.: 18/865,690

(22) PCT Filed: May 5, 2023
(86) PCT No.: PCT/IB2023/054707

§ 371 Date: Nov. 14, 2024

(30) Foreign Application Priority Data
May 14, 2022 (IN) 202231027887

(51) Int. Cl. C07C 51/235; C01B 3/32

(52) U.S. Cl. CPC C07C 51/235; C01B 3/326; C01B 2203/0233; C01B 2203/1064; C01B 2203/1229

(57) ABSTRACT

The present invention provides a process for the production of green hydrogen gas as a source of clean energy from ethanol in addition to quantitative yields of acetic acid, catalyzed by a range of group (VIII) complexes based on variety of ligands. Group (VIII) complexes consists of a series of new pincer ruthenium complexes. All of the catalytic complexes are employed towards catalytic ethanol reforming for generation of green hydrogen. Ethanol has been used as an efficient and practical hydrogen storage material. Both hydrogen and acetic acid has immense market value.

### FIELD OF THE INVENTION

The present invention relates to generation of green hydrogen and acetic acid from ethanol. More particularly, the present invention relates to the generation of green hydrogen gas as a source of clean energy from ethanol in addition to quantitative yields of acetic acid, catalyzed by a range of group (VIII) complexes based on a variety of ligands.

### BACKGROUND OF THE INVENTION

Dehydrogenation of alcohols is an efficient and clean way to generate pure hydrogen gas. There have been several reports on the generation of green hydrogen by dehydrogenation of alcohols like methanol, ethanol, isopropanol and others. Although many scientific groups have employed themselves towards the dehydrogenation of methanol, reports focusing on ethanol dehydrogenation to generate acetic acid and hydrogen is few. Most of the ethanol dehydrogenation reactions focus on the production of biofuels or on synthesis of ethyl acetate.

Acetic acid as an industrial reagent is gaining significance and its annual consumption reached 2.5 megatons in 2008 in the USA. It is used to synthesize vinyl acetate, which in turn is converted to polyvinyl acetate via polymerization. It is also used to generate esters, a significant one among these being acetyl cellulose, used as a flame retardant and environmental-friendly resin. It is also largely used as a solvent in the synthesis of polyethylene terephthalate (PET). Production of acetic acid from ethanol would be beneficial due to: i) the ready availability of bio-ethanol from cellulose, ii) efficient and environment-friendliness as it mitigates the use of CO gas (as in Monsanto or Cativa processes) and iii) the by-product of this reaction if green hydrogen, which is emerging as a promising fuel source, and ethanol can be considered as a an efficient LOHC (liquid organic hydrogen carrier). The reaction involves the following two steps:

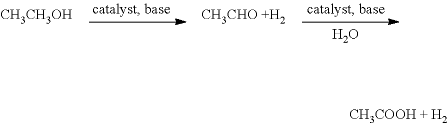

Beller et al., (ChemSusChem 2014, 7 (9), 2419-22) reported number of ruthenium and iridium-based complexes for the homogeneous transformation of bioethanol to acetic acid and hydrogen.8 All the pincer-ruthenium complexes exhibited satisfactory catalytic activity for the dehydrogenation, among which MACHO-based dihydride complex 6 and hydrochloride complex 7 showed best results up to 70% selective conversion of ethanol to acetic acid with TON up to 80,000.

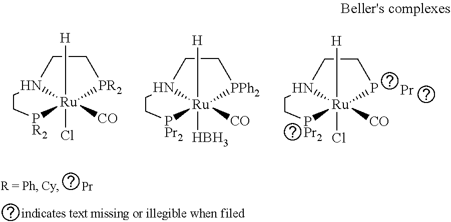

Fujita et al., (ChemCatChem 2018, 10 (17), 3636-3640) reported a series of iridium catalysts bearing a bipyriodonate ligand for dehydrogenation of ethanol-water to generate acetic acid. High yields (98%) of acetic acid were obtained along with pure hydrogen gas. The catalyst system was active under low base loading (0.6 M NaOH) and the work was also applied for dehydrogenation of several primary alcohols to their corresponding carboxylic acids.

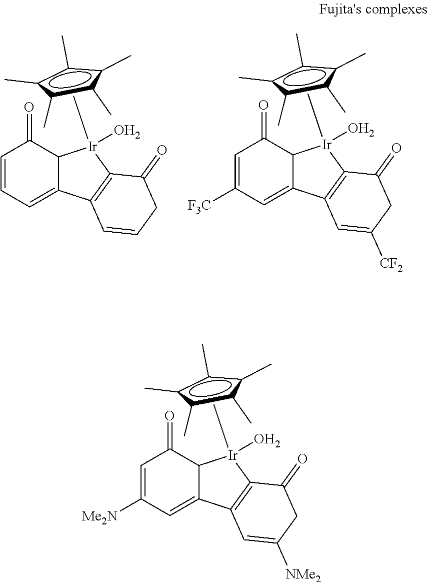

Medeiros et al., (Catal. Lett. 2000, 69 (1), 79-82) discloses the role of water in the oxidation of ethanol, showing that it decreased ethanol conversion but increased acetic acid production, catalyzed by SnO₂-supported molybdenum oxides.

Rahman et al., (Top. Catal. 2016, 59 (1), 37-45) discloses the role of water in oxidizing acetaldehyde to acetic acid and preventing the formation of crotonaldehyde was elucidated.

Xiang et al., (RSC advances 2017, 7 (61), 38586-38593) reported the same reaction via dehydrogenation-aldehyde-water shift reaction of ethanol by CuCr catalysts in an attempt to use base metals for cost effectiveness and facile operational process.

Ethanol reforming have been reported by a number of groups by using homogenous pincer complexes, as ethanol can be used as an efficient hydrogen storage medium giving high hydrogen conversion and TON. The current state of art focuses mainly on ethanol reforming to green hydrogen and acetic acid at drastic conditions.

None of the prior art discloses ethanol reforming to green hydrogen and acetic acid at ambient conditions. In light of the above, there exists a need to explore a new process for synthesizing series of new pincer-ruthenium complexes towards catalytic ethanol reforming for generation of green hydrogen. The present invention is an endeavor in this direction.

### OBJECT OF THE INVENTION

The main object of the present invention is to provide a process for generation of green hydrogen from ethanol.

Another object of the present invention is to provide a method for generation of green hydrogen gas as a source of clean energy from ethanol in addition to quantitative yields of acetic acid.

Yet another object of the present invention is to provide a method for synthesizing a series of new pincer group (VIII) complexes using appropriate metal salt with ^{R2}NNN ligand (R=^{t}Bu, ⁱPr, Cy, Ph).

Yet another object of the present invention is to provide a process for employing pincer group (VIII) complex catalyst towards catalytic ethanol reforming for generation of green hydrogen.

Still, another object of the present invention is to provide a group (VIII) catalysts mechanistic pathway for generation of green hydrogen that is used as a clean fuel. Both green hydrogen and acetic acid has immense market value.

### SUMMARY OF THE INVENTION

The present invention provides a process for the generation of green hydrogen gas as a source of clean energy from ethanol in addition to quantitative yields of acetic acid, catalyzed by a range of group (VIII) complexes based on a variety of ligands.

In an embodiment, the present invention provides a process for generation of green hydrogen from ethanol reforming, comprising the steps of: a) reacting a base with a predetermined amount of a catalyst in a reaction vessel to obtain a first reaction mixture; b) adding preweighed amount of dry ethanol and distilled water under gaseous atmosphere to the reaction mixture of step (a) to obtain a second reaction mixture; c) heating the second reaction mixture obtained in step (b) in a pre-heated oil bath at a predetermined temperature for 36-48 hours and quantifying the gas by the use of burette; and d) analyzing the composition of the gas by gas chromatography (GC) and calculating the yields of gas and acetic acid; wherein, said catalyst is pincer group (VIII) complex catalyst; the predetermined amount of the catalyst of step (a) is 0.130 g, 1.158 mmol or 0.390 g, 3.48 mmol; the predetermined amount of base of step (a) is in a range of 0.2-1.5 mol % (0.0034 g; 4.64 μmol); the gas atmosphere of step (b) is argon atmosphere and the preweighed amount of dry ethanol and distilled water of step (b) is 0.271 ml, 4.635 mmol and 0.042 ml, 2.317 mmol, respectively; and the pre-determined temperature of the oil-bath of step (c) is in a range of 110-130° C.

Here, the base is selected from sodium hydroxide, potassium hydroxide, sodium tertiary butoxide, potassium tertiary butoxide, sodium ethoxide, sodium carbonate, Cs₂CO₃, sodium, and NaHCO₃ and the yield of hydrogen is in a range of 73%-100%.

The yield of acetic acid is in a range of 73%-100% and the yield of acetic acid increase from 34.5% to 100% upon increasing the base loading to 1.0 equivalent and 1.5 equivalent of KO^{t}Bu.

The pincer group (VIII) complex catalyst for generation of green hydrogen from ethanol reforming is selected from:

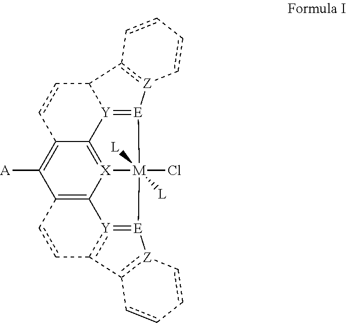

wherein,
M is selected from group (VIII) elements including iron (Fe), ruthenium (Ru), osmium (Os) and hassium (Hs);
X is selected from carbon, nitrogen or oxygen;
E is nitrogen, oxygen, phosphorus or arsenic;
A is selected from hydrogen or hydroxide;
L is selected from chlorine or triphenylphosphine;
Y is selected from methylene, oxygen, amine group or sulphur;
Z is selected from alky or aryl group; and
R is selected from the group of tertiary butyl (^{t}Bu), isopropyl (ⁱPr), cyclohexyl (Cy), hydrogen (H), methyl (CH₃) or phenyl (Ph).

In yet another embodiment of the present invention, a mechanistic pathway for the reaction to generate hydrogen gas is provided.

The present invention relates to the process for the generation of green hydrogen gas as a source of clean energy.

The above objects and advantages of the present invention will become apparent from the hereinafter set forth brief description of the drawings, detailed description of the invention, and claims appended herewith.

### BRIEF DESCRIPTION OF THE DRAWINGS

An understanding of the process for the generation of green hydrogen from ethanol through mechanistic pathway of the present invention may be obtained by reference to the following drawings:

FIG. 1 illustrates a schematic presentation of mechanistic cycle for catalytic ethanol reforming according to an embodiment of the present invention.

### DETAILED DESCRIPTION OF THE INVENTION

The present invention now will be described hereinafter with reference to the detailed description, in which some, but not all embodiments of the invention are indicated. Indeed, the invention may be embodied in many different forms and should not be construed as limited to the embodiments set forth herein; rather, these embodiments are provided so that this disclosure will satisfy applicable legal requirements. Like numbers refer to like elements throughout. The present invention is described fully herein with non-limiting embodiments and exemplary experimentation.

The present invention provides a process for the generation of green hydrogen gas as a source of clean energy from ethanol in addition to quantitative yields of acetic acid, catalyzed by a range of group (VIII) complexes based on a variety of ligands.

In a preferred embodiment, the present invention provides a process for generation of green hydrogen from ethanol reforming, comprising the steps of: a) reacting a base with a predetermined amount of a catalyst in a reaction vessel to obtain a first reaction mixture; b) adding preweighed amount of dry ethanol and distilled water under gaseous atmosphere to the reaction mixture of step (a) to obtain a second reaction mixture; c) heating the second reaction mixture obtained in step (b) in a pre-heated oil bath at a predetermined temperature for 36-48 hours and quantifying the gas by the use of burette; and d) analyzing the composition of the gas by gas chromatography (GC) and calculating the yields of gas and acetic acid; wherein, said catalyst is pincer group (VIII) complex catalyst; the predetermined amount of the catalyst of step (a) is 0.130 g, 1.158 mmol or 0.390 g, 3.48 mmol; the predetermined amount of base of step (a) is in a range of 0.2-1.5 mol % (0.0034 g; 4.64 μmol); the gas atmosphere of step (b) is argon atmosphere and the preweighed amount of dry ethanol and distilled water of step (b) is 0.271 ml, 4.635 mmol and 0.042 ml, 2.317 mmol, respectively; and the pre-determined temperature of the oil-bath of step (c) is in a range of 110-130° C.

Here, the base is selected from sodium hydroxide, potassium hydroxide, sodium tertiary butoxide, potassium tertiary butoxide, sodium ethoxide, sodium carbonate, Cs₂CO₃, sodium, and NaHCO₃ and wherein the yield of hydrogen is in a range of 73%.

The yield of acetic acid is in a range of 73%-100% and the yield of acetic acid increase from 34.5% to 73%-100% upon increasing the base loading to 1.0 equivalent and 1.5 equivalent of KO^{t}Bu.

In another embodiment, the present invention provides a series of new pincer group (VIII) complex catalyst, synthesized by using appropriate metal salt with ^{R2}NNN ligand (R=^{t}Bu, ⁱPr, Cy, Ph) in presence of CH₃CN as solvent, and all of these complexes were employed towards catalytic ethanol reforming for generation of green hydrogen.

The pincer group (VIII) complex catalyst for generation of green hydrogen from ethanol reforming is selected from:

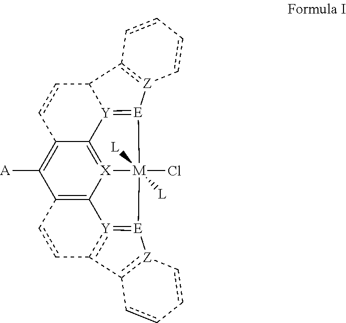

wherein,
M is selected from group (VIII) elements including iron (Fe), ruthenium (Ru), osmium (Os) and hassium (Hs);
X is selected from carbon, nitrogen or oxygen;
E is nitrogen, oxygen, phosphorus or arsenic;
A is selected from hydrogen or hydroxide;
L is selected from chlorine or triphenylphosphine;
Y is selected from methylene, oxygen, amine group or sulphur;
Z is selected from alky or aryl group; and
R is selected from the group of tertiary butyl, isopropyl, cyclohexyl, hydrogen, methyl or phenyl.

In another preferred embodiment of the present invention is provided a series of new pincer group (VIII) complex catalyst, synthesized by using appropriate metal salt with ^{R2}NNN ligand (R=^{t}Bu, ⁱPr, Cy, H, Me, Ph) in presence of CH₃CN as solvent, and all of these complexes were employed towards catalytic ethanol reforming for generation of green hydrogen. The proposed catalysts are as follows:

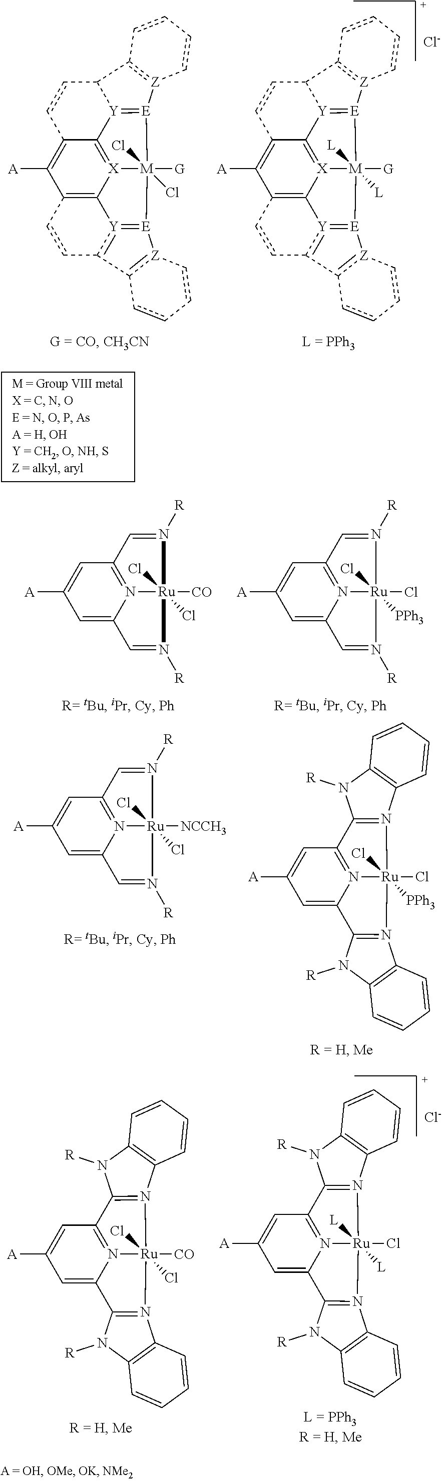

Here, R is selected from tertiary butyl (^{t}Bu), isopropyl (ⁱPr), cyclohexyl (Cy), hydrogen (H), methyl (CH₃) or phenyl (Ph).

In another embodiment, the present invention provides a mechanistic pathway for the reaction to generate hydrogen gas.

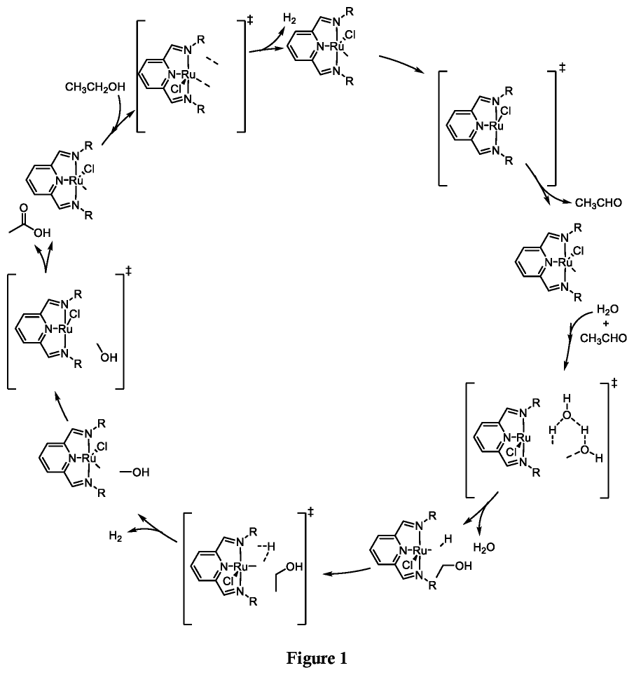

Referring to FIG. 1 of the present invention, a mechanistic pathway for the reaction is showed based on the evidence of NMR studies and formation of intermediate. The mechanistic pathway is as follows:

The first step involves the loss of PPh₃ from the complex to afford 16-electron penta-coordinate species consisting of two chloride ligands. The dichloride species engages in salt-metathesis with ethanol and base to form Ru-methoxide species. The β-H elimination in Ru-methoxide species leads to the generation of acetaldehyde and Ru—H species. The acetaldehyde formed above then reacts with water to form ethanediol via a 6-membered transition state involving two water molecules. This is followed by the σ-bond metathesis of the O—H bond bound to the metal center and Ru—H bond, liberating the first molecule of H₂. Further β-H elimination takes place that converts ethanediol to acetic acid giving back the Ru—H species. This intermediate then regenerates Ru-methoxide species via σ-bond metathesis with an additional ethanol molecule.

### Example 1

### Synthesis

### Materials and Methods

The experiment was carried out under purified AR using a standard double manifold or a glove box. The solvents such as tetrahydrofuran (THF), hexane and toluene were dried via double distillation over Na/Benzophenone prior to experiment. Ethanol was dried and distilled under argon. All other chemicals such as pyridine-2,6-dicarboxylic acid, [RuCl₂ (p-cymene)]₂, D₂O and, CDCl₃ were purchased from MERCK or Sigma-Aldrich and used as such. All catalytic reactions were set up out under an argon atmosphere using dried glassware.

### General Procedure for the Aqueous Ethanol Reforming Reaction.

In a 5 ml pear shaped vessel attached to a condenser, KO^{t}Bu (0.130 g, 1.158 mmol or 0.390 g, 3.48 mmol) and [M] (0.2 mol %; 0.0034 g; 4.64 μmol) were added inside the glove box. This was followed by addition of dry ethanol (0.271 ml, 4.635 mmol) and distilled water (0.042 ml, 2.317 mmol) under Ar atmosphere. The mixture was heated in a pre-heated oil bath at 120° C. and the gas was quantified by the use of burette, and the composition of the gas generated was analyzed by GC analysis. The reaction was run till the no more evolution of gas was observed (typically 36 h) and was then cooled down to room temperature. An aliquot (10 mg approx.) was withdrawn from reaction mixture and the NMR yield of the acetic acid was determined by ¹H NMR using D₂O as solvent and dimethyl sulfoxide as internal standard (known amount added in the flask).

### Optimization of Reaction Conditions for Ethanol Reforming:

For optimizing the reaction conditions, the influence of base on the product yield was first evaluated and the results are summarized in Table 1.

In the initial optimization, EtOH and H₂O (in 2:1 ratio) in presence of 0.5 equivalent base KO^{t}Bu and 0.2 mol % of (^{R2}NNN) RuCl₂ (PPh₃) as catalyst (entry 1, table 1) were mixed. Lower yields were obtained with NaO^{t}Bu (entry 2, table 1) and further, lower yields were obtained with KOH, NaOH, NaOEt, Na₂CO₃, Cs₂CO₃ and NaHCO₃ (entries 2-7 and entry 9, table 1). The use of sodium metal as base gave lower yield as compared to KO^{t}Bu (entry 1 vs entry 8, table 1). Upon increasing the base loading to 1.0 equivalent and 1.5 equivalent of KO^{t}Bu, 34.5% and 73% of acetic acid were obtained, respectively (entry 10 and entry 11, table 1).

TABLE 1
Variation of base in ethanol reforming with (^{R2}NNN)RuCl₂(PPh₃)^{a}

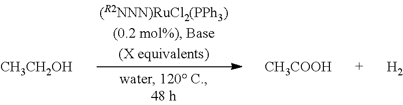

| | Base | | Yield of H₂^{b} | Yield of |
|---|---|---|---|---|
| S. No. | (X equivalents) | mmols of H₂ | (%) | CH₃COOH^{c} (%) |
| 1 | KO^{t}Bu (0.5) | 0.9 | 19 | 19 |
| 2 | NaO^{t}Bu (0.5) | 0.5 | 11 | 11 |
| 3 | KOH (0.5) | 0.6 | 12 | 12 |
| 4 | NaOH (0.5) | 0.02 | 0.4 | 0.4 |
| 5 | NaOEt (0.5) | 0.3 | 7 | 7 |
| 6 | Na₂CO₃ (0.5) | 0.005 | 0.1 | 0.1 |
| 7 | Cs₂CO₃ (0.5) | 0.01 | 0.3 | 0.3 |
| 8 | Na (0.5) | 0.9 | 15 | 15 |
| 9 | NaHCO₃ (0.5) | 0.04 | 1 | 1 |
| 10 | KO^{t}Bu (1) | 1.6 | 35 | 35 |
| 11 | KO^{t}Bu (1.5) | 3.4 | 73-100 | 73-100 |

### Reaction Condition for Hydrogen Generation:

Ethanol (0.271 ml, 4.64 mmol), H₂O (0.042 ml, 2.32 mmol), base (X equivalents), [M] (0.2 mol %) at 120° C. Gas evolution was determined by burette measurements.

Yield was calculated as n (H₂)/n (H₂O); (n (H₂) was calculated using ideal gas equation.

The yield of acetic acid was calculated by ¹H NMR spectroscopy using dimethyl sulfoxide as an internal standard.

### Physical Measurements

¹H, ²H, ¹³C{H}, ³¹P NMR were recorded on a Bruker ASCEND 600 operating at 600 MHz for ¹H and 150 MHz for ¹³C {H} or Bruker AVANCE 400 operating at 400 MHz for 1H, 100 MHz for ¹³C {H} or on a Bruker AVANCE 500 operating at 500 MHz for 1H and 125 MHz for ¹³C {H}. Chemical shifts (δ) are reported in ppm, spin-spin coupling constant (J) are expressed in Hz, and other data are reported as follows: s=singlet, d=doublet, t=triplet, m=multiplet, q=quartet, and br s=broad singlet. GC analyses were performed on a Agilent 7820-GC instrument fitted with Agilent Front SS7 inlet N2 HP-PLOT Q column (30 m length×530 μm×40 μm) using the following method: Agilent 7820-GC back detector: TCD starting temperature: 40° C.; Time at starting temp: 0 min; Ramp: 40° C./min. up to 250° C. with hold time=10 min.; Flow rate (carrier): 25 mL/min (N₂); Split ratio: 195; Inlet temperature: 40° C.; Detector temperature: TCD: 250° C., FID: 250° C.

### Example 2

### Results and Discussion

An initial optimization of this reaction was carried out with a combination of pincer-ruthenium complexes and bases in order to achieve high yields and turnovers of H₂ gas. The results of the experiments are discussed in the Table 2.

TABLE 2
H₂ production from ethanol catalysed by pincer-ruthenium complexes

| | | Catalyst | Base | mmols of | Yield of | |
|---|---|---|---|---|---|---|
| S. No. | EtOH:H₂O | (mol %) | (equiv) | gas | H₂ | TON |
| 1 | 2:1 | ^{R2}NNNRuCl₂(CO) | NaO^{t}Bu | 0.58 | 8.3 | 322 |
| | | (0.04) | (1.0) | | | |
| 2 | 2:1 | ^{R2}NNNRuCl₂(PPh₃) | KO^{t}Bu | 0.95 | 20.5 | 421 |
| | | (0.1) | (1.5) | | | |
| 3 | 1:1 | ^{R2}NNNRuCl₂(PPh₃) | KO^{t}Bu | 0.6 | 6.3 | 258 |
| | | (0.05) | (0.75) | | | |
| 4 | 1:1 | ^{R2}NNNRuCl₂(PPh₃) | KO^{t}Bu | 0.87 | 18.8 | 387 |
| | | (0.1) | (1.5) | | | |

Therefore, the present invention provides the generation of green hydrogen gas as a source of clean energy from ethanol in addition to quantitative yields of acetic acid, catalyzed by a range of group (VIII) complexes based on a variety of ligands. Herein, ethanol has been used an efficient and practical hydrogen storage material. Further, ethanol reforming to green hydrogen and acetic acid at ambient conditions.

Many modifications and other embodiments of the invention set forth herein will readily occur to one skilled in the art to which the invention pertain having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. A process for generation of green hydrogen from ethanol reforming, comprising the steps of:
a) reacting a base with a predetermined amount of a catalyst in a reaction vessel to obtain a first reaction mixture;
b) adding preweighed amount of dry ethanol and distilled water under gaseous atmosphere to the reaction mixture of step (a) to obtain a second reaction mixture;
c) heating the second reaction mixture obtained in step (b) in a pre-heated oil bath at a predetermined temperature for 36-48 hours and quantifying the gas by the use of burette; and
d) analyzing the composition of the gas by gas chromatography (GC) and calculating the yields of gas and acetic acid;
wherein,
said catalyst is pincer group (VIII) complex catalyst;
the predetermined amount of the catalyst of step (a) is 0.130 g, 1.158 mmol or 0.390 g, 3.48 mmol;
the predetermined amount of base of step (a) is in a range of 0.2-1.5 mol % (0.0034 g; 4.64 μmol);
the gas atmosphere of step (b) is argon atmosphere and the preweighed amount of dry ethanol and distilled water of step (b) is 0.271 ml, 4.635 mmol and 0.042 ml, 2.317 mmol, respectively; and
the pre-determined temperature of the oil-bath of step (c) is in a range of 110-130° C.

2. The process as claimed in claim 1, wherein the base is selected from sodium hydroxide, potassium hydroxide, sodium tertiary butoxide, potassium tertiary butoxide, sodium ethoxide, sodium carbonate, Cs₂CO₃, sodium, and NaHCO₃.

3. The process as claimed in claim 1, wherein the yield of hydrogen is in a range of 73%-100%.

4. The process as claimed in claim 1, wherein the yield of acetic acid is in a range of 73%-100%.

5. The process as claimed in claim 1, wherein the yield of acetic acid increase from 34.5% to 73%-100% upon increasing the base loading to 1.0 equivalent and 1.5 equivalent of KO^{t}Bu.

6. The process as claimed in claim 1, wherein said pincer group (VIII) complex catalyst for generation of green hydrogen from ethanol reforming is selected from: 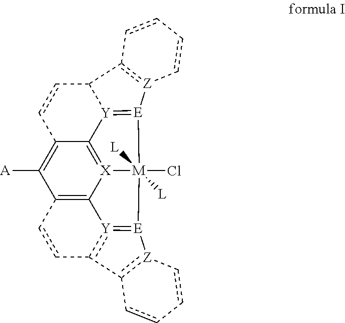
wherein,
M is selected from group (VIII) elements including iron (Fe), ruthenium (Ru), osmium (Os) and hassium (Hs);
X is selected from carbon, nitrogen or oxygen;
E is nitrogen, oxygen, phosphorus or arsenic;
A is selected from hydrogen or hydroxide or alkoxide;
L is selected from chlorine or triphenylphosphine;
Y is selected from methylene, oxygen, amine group or sulphur;
Z is selected from alky or aryl group; and
R is selected from the group of tertiary butyl (^{t}Bu), isopropyl (ⁱPr), cyclohexyl (Cy), hydrogen (H), methyl (CH₃) or phenyl (Ph).
